(19) United States
(12) Patent Application Publication
(10) Pub. No.: US 2009/0148522 A1
(43) Pub. Date: Jun. 11, 2009

(54) HEATED ROLLER COMPACTION PROCESS FOR MAKING PHARMACEUTICAL COMPOSITIONS

(76) Inventors: James Kowalski, Belle Mead, NJ (US); Jay Parthiban Lakshman, Cedar Knolls, NJ (US); Abu T.M. Serajuddin, Queens, NY (US); Wei-Qin Tong, Basking Ridge, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/299,036

(22) PCT Filed: May 3, 2007
(86) PCT No.: PCT/US07/10671

§ 371 Date: Oct. 30, 2008

Related U.S. Application Data
(60) Provisional application No. 60/746,407, filed on May 4, 2006.

(51) Int. Cl. A61K 9/20

(52) U.S. Cl. 424/465

(57) ABSTRACT

A process for using a heated roller compactor to prepare melt granulated composition of a therapeutic compound, especially a poorly compressible and/or moisture sensitive therapeutic compound, with a granulation excipient.

### FIELD OF THE INVENTION

The present invention relates to a process for making solid oral dosage forms of a poorly compressible and/or a moisture sensitive therapeutic compound. The process features the use of a heated roller compactor.

### BACKGROUND OF THE INVENTION

Poor compressibility can impact the ability of formulating a therapeutic compound into a solid oral dosage form, e.g., a tablet. Conventional tablet formulations containing poorly compressible therapeutic compounds often lack adequate hardness and are friable. Thus, special formulation techniques are used to formulate poorly compressible therapeutic compounds into commercially viable solid oral dosage forms, especially tablets.

One way to overcome the poor compressibility of therapeutic compounds is to utilize wet granulation techniques to prepare the tablet formulation. This involves additional unit operations of wet milling, drying and milling of dried granulation. However, some tablets prepared by wet methods can show incremental hardness as a function of time and storage temperature. Therefore, tablets prepared by wet methods can show variable product performance. Additionally, certain therapeutic compounds are susceptible to degradation when in contact with water; thus, wet granulation with water may not be ideal.

Thus, there is a need for a method of preparing pharmaceutical compositions of poorly compressible therapeutic compounds that have adequate hardness with good reproducibility. This invention addresses that need by utilizing melt granulation techniques. A particularly inventive aspect of the present invention is the use of a heated roller compactor for melt granulation compounding.

Traditionally, roller compactors have been used for dry granulation processes. A roller compactor forces fine powders between rotating rolls in order to compress the powders into a smaller volume forming a compact or sheet. The present invention expands the use of roller compactors such that they are appropriate for melt granulating pharmaceutical compositions.

### SUMMARY OF THE INVENTION

The present invention features a process for making a pharmaceutical composition that includes the steps of combining a poorly compressible and/or moisture sensitive therapeutic compound with at least one granulation excipient to form a mixture; compressing the mixture in roller compactor that is heated to a temperature less than the melting point or melting range of the therapeutic compound.

In a particular aspect, the compact can be optionally milled into granules and subsequently compressed using conventional means into a solid oral dosage form. In another aspect of the present invention, the granulation excipient is a polymer having a glass transition temperature that is less than the melting point of the therapeutic compound. Particularly useful polymers include water-soluble, water-swellable and water insoluble polymers.

The inventive process of the present invention can be used to make both immediate release and sustained release pharmaceutical compositions.

### BRIEF DESCRIPTION OF THE DRAWINGS

The accompanying drawings, which are incorporated in and constitute a part of the specification, illustrate an exemplary embodiment of the present invention.

FIG. 1 shows a sectional view of an exemplary embodiment of a roller compactor;

FIG. 2. shows a side view of an exemplary embodiment of rollers in a heated roller compactor that is heated by electrical heating element;

FIG. 3. is a side view of one of the rolls in FIG. 2; and

FIG. 4 shows a side view of an exemplary embodiment of rollers in a heated roller compactor that is heated by fluid means.

### DETAILED DESCRIPTION OF THE INVENTION

The present invention relates to a process for preparing pharmaceutical compositions of poorly compressible and/or moisture sensitive therapeutic compounds. The inventive process features melt granulation, using a heated roller compactor, of a poorly compressible therapeutic compound with a granulation excipient. The melt granulation of the poorly compressible therapeutic compound is accomplished without the need for any melting of the therapeutic compound.

As used herein the term “pharmaceutical composition” means a mixture containing a therapeutic compound to be administered to a mammal, e.g., a human in order to prevent, treat or control a particular disease or condition affecting the mammal.

As used herein the term “pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

As used herein the term “therapeutic compound” means any compound, substance, drug, medicament, or active ingredient having a therapeutic or pharmacological effect, and which is suitable for administration to a mammal, e.g., a human, in a composition that is particularly suitable for oral administration.

As used herein the term “poorly compressible” therapeutic compound refers to a compound that does not easily bond to form a tablet upon the application of a force. A tablet produced solely of the therapeutic compound weighing one gram and compressed under a force ranging from 5 kN to 25 kN with a dwell time under thirty seconds, would provide friability at or above an acceptable limit of 1.0% (w/w) when tablets weighing approximately ten grams (or at least ten units) are tested after five hundred drops immediately after compression. Such compounds may require additional processing and special formulating, for example wet granulating or roller compacting, prior to compression. High dosages of a therapeutic compound may also render a therapeutic compound not appropriate for direct compression because of poor flowability and poor compressibility.

As used herein, the term “moisture-sensitive” therapeutic compound refers to a therapeutic compound which undergoes degradation during or after preparation of the tablet, e.g., by hydrolysis of at least 1% by weight of the therapeutic compound.

The poorly compressible therapeutic compound(s) is present in the pharmaceutical compositions of the present invention in a therapeutically effective amount or concentration. Such a therapeutically effective amount or concentration is known to one of ordinary skill in the art as the amount or concentration varies with the therapeutic compound being used and the indication which is being addressed. For example, in accordance with the present invention, the therapeutic compound may be present in an amount by weight of about 0.05% to about 99% weight of pharmaceutical composition. In one embodiment, the therapeutic compound may be present in an amount by weight of about 10% to about 95% by weight of the pharmaceutical composition.

As used herein, the term “immediate release” refers to the rapid release of the majority of the therapeutic compound, e.g., greater than about 50%, about 60%, about 70%, about 80%, or about 90% within a relatively short time, e.g., within 1 hour, 40 minutes, 30 minutes or 20 minutes after oral ingestion. Particularly useful conditions for immediate-release are release of at least or equal to about 80% of the therapeutic compound within thirty minutes after oral ingestion. The particular immediate release conditions for a specific therapeutic compound will be recognized or known by one of ordinary skill in the art.

As used herein, the term “sustained release”, or modified release, refers to the gradual but continuous or sustained release over a relatively extended period of the therapeutic compound content after oral ingestion. The release will continue over a period of time and may continue through until and after the pharmaceutical composition reaches the intestine. Sustained release may also refer to delayed release in which release of the therapeutic compound does not start immediately when the pharmaceutical composition reaches the stomach but is delayed for a period of time, for instance, until when the pharmaceutical composition reaches the intestine when the increasing pH is used to trigger release of the therapeutic compound from the pharmaceutical composition. Such aforementioned release profiles can be achieved by the use of a release retardant as set forth below.

As used herein the term “release retardant” refers to any material or substance that slows the release of a therapeutic compound from a pharmaceutical composition when orally ingested. Such release retardants can provide a sustained or modified release propfile. Various sustained release systems, as known in the art, can be accomplished by the use of a release retarding component, e.g., a diffusion system, a dissolution system and/or an osmotic system. A release retardant can be polymeric or non-polymeric in nature. The pharmaceutical compositions of the present invention can include, for example, at least five percent of a release retardant by weight of the composition if a sustained release composition is desired. Release retardants can include, but are not limited to, any of the granulation excipients as defined below.

As used herein the term “granulation excipient” refers to any pharmaceutically acceptable material or substance that can be melt granulated with the poorly compressible therapeutic compound as further described below. The granulation excipient, for example, can be a polymer or a non-polymeric material.

As used herein the term “polymer” refers to a polymer or mixture of polymers that have a glass transition temperature, softening temperature or melting temperature by itself or in combination not exceeding the melting point (or melting range) of the poorly compressible therapeutic compound. The glass transition temperature (“Tg”) is the temperature at which such polymer's characteristics change from that of highly viscous to that of relatively less viscous mass. Types of polymers include, but are not limited to, water-soluble, water-swellable, water insoluble polymers and combinations of the foregoing.

Examples of polymers include, but are not limited to:

homopolymers and copolymers of N-vinyl lactams, e.g., homopolymers and copolymers of N-vinyl pyrrolidone (e.g., polyvinylpyrrolidone), copolymers of N-vinyl pyrrolidone and vinyl acetate or vinyl propionate;

cellulose esters and cellulose ethers (e.g., methylcellulose and ethylcellulose) hydroxyalkylcelluloses (e.g., hydroxypropylcellulose), hydroxyalkylalkylcelluloses (e.g., hydroxypropylmethylcellulose), cellulose phthalates (e.g., cellulose acetate phthalate and hydroxylpropylmethylcellulose phthalate) and cellulose succinates (e.g., hydroxypropylmethylcellulose succinate or hydroxypropylmethylcellulose acetate succinate);

high molecular polyalkylene oxides such as polyethylene oxide and polypropylene oxide and copolymers of ethylene oxide and propylene oxide;

polyacrylates and polymethacrylates (e.g., methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates));

polyacrylamides;

vinyl acetate polymers such as copolymers of vinyl acetate and crotonic acid, partially hydrolyzed polyvinyl acetate;

polyvinyl alcohol; and

oligo- and polysaccharides such as carrageenans, galactomannans and xanthan gum, or mixtures of one or more thereof.

As used herein, the term “plasticizer” refers to a material that may be incorporated into the pharmaceutical composition in order to decrease the glass transition temperature and the melt viscosity of a polymer by increasing the free volume between polymer chains. Plasticizers, for example, include, but are not limited to, water; citrate esters (e.g., triethylcitrate, triacetin); low molecular weight poly(alkylene oxides) (e.g., poly(ethylene glycols), poly(propylene glycols), poly(ethylene/propylene glycols)); glycerol, pentaerythritol, glycerol monoacetate, diacetate or triacetate; propylene glycol; sodium diethyl sulfosuccinate; and the therapeutic compound itself. The plasticizer can be present in concentration from about 0% to 15%, e.g., 0.5% to 5% by weight of the pharmaceutical composition. Examples of plasticizers can also be found in The Handbook of Pharmaceutical Additives, Ash et al., Gower Publishing (2000).

Non-polymeric granulation excipients include, but are not limited to, esters, hydrogenated oils, oils, natural waxes, synthetic waxes, hydrocarbons, fatty alcohols, fatty acids, monoglycerides, diglycerides, triglycerides and mixtures thereof.

Examples of esters, such as glyceryl esters include, but are not limited to, glyceryl monostearate, e.g., CAPMUL GMS from Abitec Corp. (Columbus, Ohio); glyceryl palmitostearate; acetylated glycerol monostearate; sorbitan monostearate, e.g., ARLACEL 60 from Uniqema (New Castle, Del.); and cetyl palmitate, e.g., CUTINA CP from Cognis Corp. (Dusseldorf, Germany), magnesium stearate and calcium stearate.

Examples of hydrogenated oils include, but are not limited to, hydrogenated castor oil; hydrogenated cottonseed oil; hydrogenated soybean oil; and hydrogenated palm oil. An example of oil include sesame oil.

Examples of waxes include, but are not limited to, carnauba wax, beeswax and spermaceti wax. Examples of hydrocarbons include, but are not limited to, microcrystalline wax and paraffin. Examples of fatty alcohols, i.e., higher molecular weight nonvolatile alcohols that have from about 14 to about 31 carbon atoms include, but are not limited to, cetyl alcohol, e.g., CRODACOL C-70 from Croda Corp. (Edison, N.J.); stearyl alcohol, e.g., CRODACOL S-95 from Croda Corp; lauryl alcohol; and myristyl alcohol. Examples of fatty acids which may have from about 10 to about 22 carbon atoms include, but are not limited to, stearic acid, e.g., HYSTRENE 5016 from Crompton Corp. (Middlebury, Conn.); decanoic acid; palmitic acid; lauric acid; and myristic acid.

As used herein, the term “melt granulation” refers to the following compounding process that comprises the steps of:
(a) forming a mixture of a poorly compressible therapeutic compound with at least one granulation excipient;
(b) granulating the mixture using a roller compactor that has its rollers heated to a temperature that is less than or about at the melting point (or melting range) of the poorly compressible therapeutic compound; and
(c) cooling the extrudate to room temperature, for example, at a controlled rate.

The heating and mixing of the therapeutic compound and the granulation excipient to form an internal phase of granules (i.e., from the extrudate) is accomplished by the use of an extruder. The granulation excipient, e.g., can be present in an amount from about 1% to about 50% by weight of the composition. In one embodiment, the granulation excipient may be present in an amount from about 3 to about 25% by weight of the composition. The therapeutic compound may be present in an amount from about 50% to about 99% by weight of the composition. In one embodiment, the therapeutic compound may be present in an amount of about 60% to about 97%.

The resulting granules are, for example, particles of the therapeutic compound coated or substantially coated by the granulation excipient, forming a “melt layer”, or alternatively, particles of the therapeutic compound embedded or substantially embedded with or within the granulation excipient. This can increase compactibility of the drug substance and, depending on excipient(s) used, can also retard drug release to form slow-release products.

Additionally, such a melt layer can be useful for acting as a barrier against a physical or chemical incompatibility between ingredients within a formulation, for example between or among the therapeutic compound and excipients or between multiple therapeutic compounds in a combination. For example, at times, multiple therapeutic compounds may be incompatible with each other. Examples include, but are not limited to, combinations of reactive materials (e.g., acids and bases, oxidizers and reducers, organic acids and alcohols); and/or combinations of physically incompatible materials (e.g., eutectic forming, water-mediated, i.e., one therapeutic compound absorbs moisture thus introducing water, and the other is instable in the presence of water).

In general, the roller compactor equipment includes rollers, for example two counter rotating rolls. As the rolls turn towards each other, material is fed into the nip area formed between the surfaces of the rolls. The reduction of volume and the pressure from the nip region causes the material to form a solid compact or sheet. The duration that the material is compacted between the rolls is known as residence time. An example of a roller compactor suitable for use in the present invention is equipment from the CHILSONATOR® series from The Fitzpatrick Company (Elmhurst, Ill.).

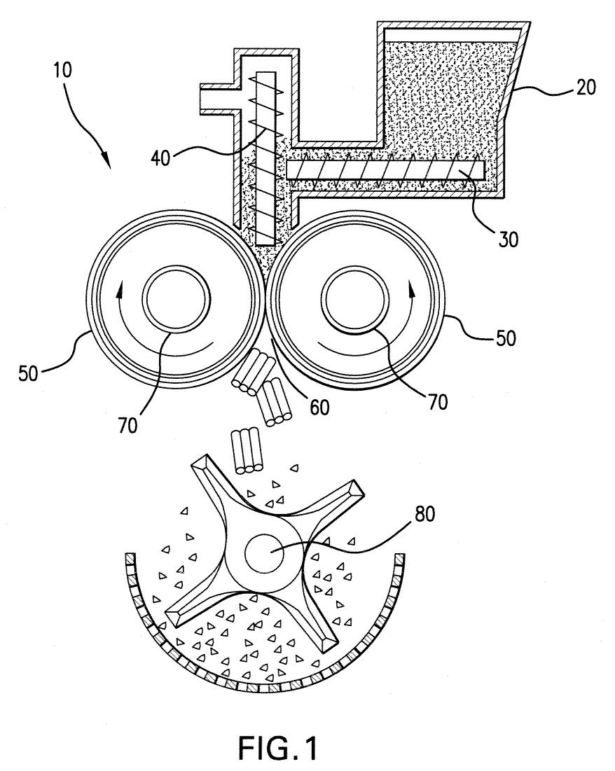

FIG. 1 is a schematic showing the parts of an exemplary roller compactor 10 as commonly known in the art. The materials, e.g. the therapeutic compound and any granulation excipients, is first added to a hopper 20. The material is then transferred along a horizontal metering screw 30 to a vertical deaerating, precompression screw 40. Material transfers from the precompression screw 40 into the nip region 60 created between the counter-rotating rollers 50. The rollers 50 are physically driven by the drive shafts 70. The resulting granulation can be subsequently comminuted with a mill 80.

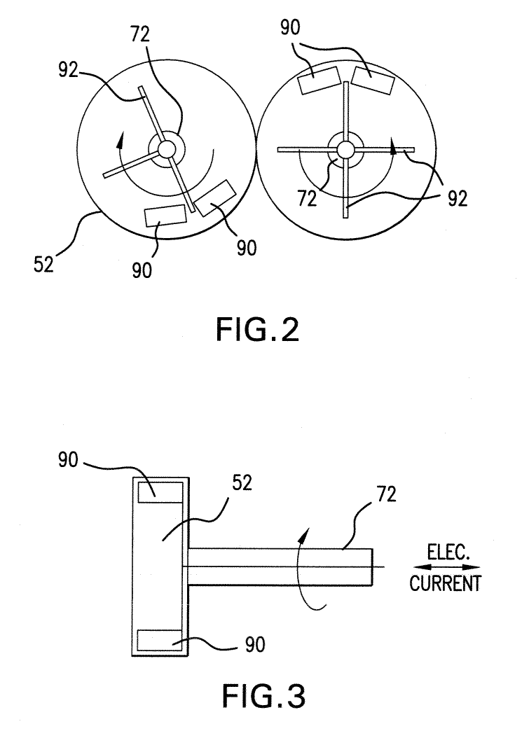

As used in the present invention, the rollers 50 of the exemplary roller compactor 10 are modified such that they may be heated. As used herein, the term “heated roller compactor” means that the rollers of the roller compactor are heated at an operating temperature greater than 40° C. FIGS. 2 and 3 show exemplary rollers 52 that are heated by heating elements 90. Such heating elements 90 can be attached via conductors 92 to the drive shafts 72 which generate electricity or are connected to an electrical source. The electricity conveyed by the drive shafts 72 and/or conductors 92 heat the heating elements 90 which in turn heat the surfaces of the rollers 52. FIG. 2 shows a side view of the exemplary rollers 52, and FIG. 3. shows a front view of a single roller.

Examples of an heating element is SAMOX Insulated heating tape commercially available from Cole Parmer (Vernon Hills, Ill.) with 156 to 1256 W each powered at 100 VAC/15 Amps. A single or multiple heating element can be used to heat the roller 50 to the required temperature.

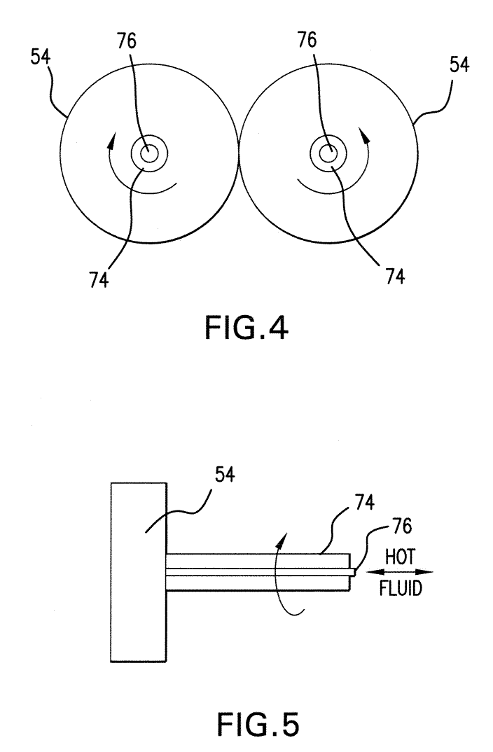

Alternatively, the rollers 50 can be heated by fluid means. FIGS. 4 and 5 show exemplary rollers 54 heated by hot fluids (e.g., hot water, steam, oil, etc.) The drive shafts 74 can be coaxial tubes such that the hot fluid is introduced into the inner tube 76 as shown in FIG. 5. The hot fluid subsequently heats the rollers 54.

A heated roller compactor offers advantages in situations in which a short residence time is needed. For example, other melt granulation techniques may have long residence times, for example greater than one minute which could lead to or cause some thermal degradation of the therapeutic compound and/or granulation excipient. With a heated roller compactor, the residence time may be as short as one to two seconds, or a few seconds in duration.

On the other hand, if a longer residence time is needed, for example, to allow a complete melting of an added excipient and/or increased coating of drug substances, the process can be modified to allow a series of roller compactors instead of just two of them.

Once the granules are obtained, the granules may be formulated into oral forms (with or without being previously milled), e.g., solid oral dosage forms, such as tablets, pills, lozenges, caplets, capsules or sachets, by adding additional pharmaceutically acceptable excipients which comprise an external phase of the pharmaceutical composition. Examples of such pharmaceutically acceptable excipients include, but are not limited to, release retardants, plasticizers, disintegrants, binders, lubricants, glidants, stabilizers, fillers and diluents. One of ordinary skill in the art may select one or more of the aforementioned excipients with respect to the particular desired properties of the solid oral dosage form by routine experimentation and without any undue burden. The amount of each excipient used may vary within ranges conventional in the art. The following references which are all hereby incorporated by reference discloses techniques and excipients used to formulate oral dosage forms. See The Handbook of Pharmaceutical Excipients, 4^{th} edition, Rowe et al., Eds., American Pharmaceuticals Association (2003); and Remington: the Science and Practice of Pharmacy, 20^{th} edition, Gennaro, Ed., Lippincoft Williams & Wilkins (2003).

Examples of pharmaceutically acceptable disintegrants include, but are not limited to, starches; clays; celluloses; alginates; gums; cross-linked polymers, e.g., cross-linked polyvinyl pyrrolidone or crospovidone, e.g. POLYPLASDONE XL from international Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 1.5% by weight of composition.

Examples of pharmaceutically acceptable binders include, but are not limited to, starches; celluloses and derivatives thereof, for example, microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder may be present in an amount from about 0% to about 50%, e.g., 10-40% by weight of the composition.

Examples of pharmaceutically acceptable lubricants and pharmaceutically acceptable glidants include, but are not limited to, colloidal silica, magnesium trisilicate, fatty acids such as stearic acid, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 15% to about 40% by weight of the composition.

To make pharmaceutical compositions of the present invention, a therapeutic compound and a granulation excipient are blended in a ratio in a range of 99:1 to 30:70 (on a dry weight basis) prior to, or upon addition into the hopper of an extruder. In one exemplary embodiment, this ratio between the therapeutic compound and granulation excipient can be in a range of 97:3 to 60:40 (on a dry weight basis). Yet in another alternative embodiment, the ratio can be in a range of 97:3 to 75:25 (on a dry weight basis). Optionally, a plasticizer can be added to the internal phase.

The mixture is heated to a temperature(s) less than the melting temperature of the therapeutic compound. As the mixture is being heated, it is also being compacted by the rollers of the roller compactor. As the mixture exits the nip region of the roller compactor, it is granulated and allowed to cool.

After cooling, the extrudate can be milled and subsequently screened through a sieve. The granules (which constitute the internal phase of the pharmaceutical composition) are then combined with solid oral dosage form excipients (the external phase of the pharmaceutical composition), i.e., fillers, binders, disintegrants, lubricants and etc. The combined mixture may be further blended, e.g., through a V-blender, and subsequently compressed or molded into a tablet, for example a monolithic tablet, or encapsulated by a capsule.

When multiple therapeutic compounds are used in the formulation, some of the therapeutic compounds can reside in the internal phase of the pharmaceutical composition, and the others can reside in the external phase. For example, with two therapeutic compounds, one therapeutic compounds can reside in each phase. In this exemplary scenario, the internal phase therapeutic compound can be coated by the granulation excipient. The second therapeutic compound is incorporated in the external phase. Thus, the granulation excipient functions as the melt layer between the internal and external phase therapeutic compounds to reduce the reactivity and/or interaction between the two therapeutic compounds.

Once the tablets are obtained, they can be optionally coated with a functional or non-functional coating as known in the art. Examples of coating techniques include, but are not limited to, sugar coating, film coating, microencapsulation and compression coating. Types of coatings include, but are not limited to, enteric coatings, sustained release coatings, controlled-release coatings.

The utility of all the pharmaceutical compositions of the present invention may be observed in standard clinical tests in, for example, known indications of drug dosages giving therapeutically effective blood levels of the therapeutic compound; for example using dosages in the range of 2.5-1500 mg of therapeutic compound per day for a 75 kg mammal, e.g., adult and in standard animal models.

The present invention provides a method of treatment of a subject suffering from a disease, condition or disorder treatable with a therapeutic compound comprising administering a therapeutically effective amount of a pharmaceutical composition of the present invention to a subject in need of such treatment.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

1. A process for making a pharmaceutical composition comprising the step of compounding a therapeutic compound with a granulation excipient in a heated roller compactor.

2. The process of claim 1, wherein said therapeutic compound is a poorly compressible compound.

3. The process of claim 1, wherein said therapeutic compound is a moisture sensitive compound.

4. A process for making a pharmaceutical composition comprising the steps of:
combining a therapeutic compound with at least one granulation excipient to form a mixture; and
compacting said mixture in a heated roller compactor to obtain said pharmaceutical composition.

5. The process of claim 4, wherein said pharmaceutical composition comprises granules.

6. The process of claim 4, wherein said pharmaceutical composition is a solid oral dosage form.

7. The process of claim 6, wherein said solid oral dosage form is a tablet.

8. The process of claim 5 further comprising the step of milling said granules.

9. The process of claim 8 further comprising the step of compressing said granules with at least one pharmaceutically acceptable excipient to form a tablet.

10. The process of claim 4, wherein said granulation excipient is selected from the group consisting of water soluble polymers, water swellable polymers and water insoluble polymers.

11. The process of claim 4, wherein said granulation excipient is a release retardant.

12. The process of claim 4, wherein said roller compactor is heated to a temperature from 40° C. to the melting point of said therapeutic compound.

13. The process of claim 5, wherein said granules comprise a melt layer between said therapeutic compound and said granulation excipient.

14-15. (canceled)
